(19) United States
(12) Patent Application Publication
(10) Pub. No.: US 2021/0027890 A1
(43) Pub. Date: Jan. 28, 2021

(54) DETECTING, EVALUATING AND PREDICTING SYSTEM FOR CANCER RISK

(71) Applicant: ConnSante BioTech, Inc., Hsinchu (TW)

(72) Inventors: Hsiao-Hui FU, Hsinchu (TW); Juinn LEE, Hsinchu (TW)

(21) Appl. No.: 16/937,226

(22) Filed: Jul. 23, 2020

Related U.S. Application Data
(60) Provisional application No. 62/878,096, filed on Jul. 24, 2019.

(51) Int. Cl. G16H 50/20; G06T 7/00; G16H 50/70; G06N 20/00; G16H 10/60

(52) U.S. Cl. CPC G16H 50/20; G06T 7/0012; G16H 10/60; G06N 20/00; G16H 50/70

(57) ABSTRACT

The present invention provides an algorithm model for determining the probability, or risk of incidence of cancer and estimating the chance that a subject with given risk factors will develop cancer over a specified interval or lifetime. The present invention provides algorithm-based molecular and cell biological assays that involve measurement of expression levels of proteins or/and genes from a biological sample obtained from a subject. The present invention also provides methods of acquiring a quantitative score based on measurement of expression levels of proteins or/and genes from a biological sample from a subject. These proteins or/and genes would be grouped into functional subsets and weighted according to their contribution to cancer risk.

### CROSS REFERENCE TO RELATED APPLICATIONS

This application claims priority in U.S. Provisional Patent Application No. 62/878,096, filed Jul. 24, 2019, which is incorporated by reference in its entirety herein.

### BACKGROUND OF INVENTION

### 1. Field of the Invention

The present invention provides a system and method combining biotechnical detection technique with cancer risk prediction machine learning model, and more particularly to an intelligent prediction system and method for early warning of cancer risk for an individual.

### 2. Description of Related Art

According to a global incidence of cancer published by the Organization for Economic Cooperation and Development (OECD), the incidence of cancer in Denmark is about 338.1 per 100,000 population, the incidence of cancer in the U.S. is about 318 per 100,000 population, the incidence of cancer in Taiwan is about 296.7 per 100,000 population. The incidences of cancer in Denmark, U.S, Taiwan were ranked first, fifth and tenth in the world respectively. The major causes for cancers include food habit, work and rest scheduling, genes and environment. However, for different races, nations and countries, various countries have very different incidences of cancer, as well as far different types of prevalent cancer.

At present, there are no extraordinary symptoms in early stage of cancer by the general public knowledge. However, once symptoms happened, the patient's condition is mostly serious. This is because the current detection technique for cancer or tumor, most of which are obtained through biomedical imaging. However, the cancer cells accumulation to be a large quantity to be detected (e.g. accumulate ˜10,000,000 cells as ˜0.2 cm³ tumor clustering), and then the individual's cancer symptoms are already in a serious state.

In view of this, the urgent problem for those of related technical fields to be solved is to develop a non-invasive detection for early detection of the “anti-cancer capability” expressed by cellular immune system and tissue repair system related body conditions for individuals, which can be extensively used for detection of various cancers, to improve the risk factors in life for the subjects early to reduce continuous accumulation of cancer risks, so as to solve the deficiencies in the existing technology.

### SUMMARY OF THE INVENTION

The purpose of the present invention is to combine biotechnical detection technique with cancer risk prediction machine learning model to provide rating of overall anti-cancer capability of a subject's body conditions, individual lifetime cancer risk analysis and estimated reference value of lifetime cancer risk, to prevent the risk of individual's tumor formation or to monitor the risk of tumor recurrence and metastasis, mastering personal health management efficiency continuously, further improving the existing technology.

The present invention provides a composite system for machine learning image evaluation and cancer risk prediction, which comprises an optical image evaluation and intelligent calibrating system and a cancer risk prediction and machine-learning system.

In an embodiment of the present invention, the optical image evaluation and intelligent calibrating system comprises an image modeling module, an image acquisition case database module and a multitask image analysis module. The image modeling module classifies the cell staining image results of GSK-3α protein expression level of a target cell into Grade A, Grade B, Grade C and Grade D to build an image detection model. The multitask image analysis module uses the image detection model built by the image modeling module to analyze the detection images in the detection results obtained by the image acquisition case database module, and the image detection model generates the corresponding analysis result.

In an embodiment of the present invention, the cancer risk prediction and machine-learning system comprises a public health database, an input module, an information acquisition module and a machine learning analysis module. The input module allows a user to input an individual's sexuality and age via a webpage interface or App programming interface, which are saved in the public health database. The information acquisition module can extract the average cancer risk of population and the cancer risk with family history of cancer/cancer risk without family history of cancer automatically, which are stored in the public health database. The information acquisition module can extract the detection image of the individual from the optical image evaluation and intelligent calibrating system automatically to generate the corresponding “anti-cancer capability”. The machine learning analysis module is communicated with the public health database, wherein the machine learning analysis module generates the corresponding analysis result according to the average cancer risk of population and/or the cancer risk with family history of cancer, cancer risk without family history of cancer, cancer patient survival status or metastasis and recurrence status, as well as the detection images from the optical image evaluation and intelligent calibrating system, to perform machine learning and to build a cancer risk prediction model, so as to obtain a cancer risk prediction table of the individual.

To make the aforesaid features and advantages of the present invention more intelligible, there are embodiments given below, and elaborated with attached figures as follows.

### BRIEF DESCRIPTION OF THE DRAWINGS

FIG. 1A-FIG. 1D is the schematic example of grading result of GSK-3α staining of target cell in an embodiment of the present invention. FIG. 1A is represented Grade A for GSK-3α staining of target cell. FIG. 1B is represented Grade B for GSK-3α staining of target cell. FIG. 1C is represented Grade C for GSK-3α staining of target cell. FIG. 1D is represented Grade D for GSK-3α staining of target cell.

FIG. 2 is the schematic diagram of cancer patient survival status or metastasis and recurrence status of cancer risk prediction machine learning model of the present invention.

FIG. 3 is the schematic diagram of a composite system of machine learning image evaluation and cancer risk prediction in an embodiment of the present invention

### DETAILED DESCRIPTION OF THE INVENTION

The technical content of the present invention is described below by specific implementation patterns, those who are familiar with this craft can understand the advantages and effects of the present invention according to the content disclosed in this specification. However, the present invention can be practiced or applied in different forms of patterns without deviating from the characteristics or spirit of the present invention.

An embodiment of the present invention in the description is used for description, representing a specific function, structure or feature involved in the present invention. The term “an embodiment” in the description is not always the same embodiment, or a specific or substitute embodiment mutually exclusive of other embodiments. In other words, some embodiments can describe some specific features, whereas other embodiments don't. In addition, the well-known structures, elements or connections of related fields are not described in detail, so as to avoid obscuring the particular features of the present invention.

In the present invention, “connection”, “electrical connection” and “communication linkage” represent interoperation or interaction of two or multiple elements.

In an embodiment of the present invention, the composite system at least comprises an optical image evaluation and intelligent calibrating system and a cancer risk prediction and machine-learning system.

In an embodiment of the present invention, the term “individual” or “subject” used in this invention refers to an organism, and more particularly to an organism without cancer record, a human body without cancer record. Wherein the specimen of the aforementioned individual can be an organism, human blood, bone marrow or tissue, wherein the tissue is any tissue obtained from the organism and human body. In addition, the aforesaid target cell is an “unhealthy cell”. Furthermore, an image or a spectrum of the aforementioned specimen can be captured and interpreted by the optical acquisition interpretation element.

In another embodiment, the aforementioned “anti-cancer capability” in the present invention is defined as GSK-3α protein expression level in the staining cells. In other words, the present invention can establish a cancer risk estimation according to the amount of GSK-3α protein expressed by individual cells and the existence or status of “unhealthy cell”, which can represent the aforementioned anti-cancer capability of the present invention.

In another embodiment of the present invention, the “lethal cell” in the present invention is defined a hematopoietic stein cells (HSC) or a mesenchymal stein cell (MSC) with an aberrant nucleus and cytoplasm accumulation of GSK-3α. In actual implementation of the present invention, the “lethal cell” is detected by selecting the mononuclear cell cluster which may contain one or a plurality of “lethal cells”.

In another embodiment of the present invention, the aforementioned “unhealthy cell” in the present invention is defined a cell associated with aberrant expression of GSK-3α, wherein the cell is selected from mesenchymal stein cell (MSC), mesenchymal progenitor cell (MPC), mesenchymal stein and progenitor cell (MSPC), hematopoietic stein cells (HSC), hematopoietic progenitor cell (HPC), hematopoietic stein and progenitor cell (HSPC), fibrocyte, macrophage, fibroblast, myofibroblast and mesenchymal cell. In actual implementation of the present invention, the “unhealthy cell” can be detected by selecting a mononuclear cell cluster which may contain one or a plurality of “unhealthy cells”.

Furthermore, in an embodiment of the present invention wherein the target cell is the mononuclear cells of individual's immune system or tissue repair system. The GSK-3α protein expression levels can be classified into Grade A, Grade B, Grade C and Grade D after staining and interpretation.

Certainly, the present invention is not limited to this, the target cell of the specimen includes a myeloid derived suppressor cell (MDSC) and a T cell, wherein the MDSC is a biomarker with Lin⁻/HLA-DR⁻/CD33⁺/CD11b⁺, herein the T cell is a cytotoxic T cell, especially a CD3⁺CD8⁺ T cell.

In an embodiment of the present invention, the optical image capture element can be disposed in a slide scanner, a microscope or a flow cytometer. Wherein the optical image capture element can obtain images with a light source. The light source can be a visible light source or a laser source. The aforementioned optical image capture element is not limited to optical camera, digital camera, video camera, optical collector and detector.

In an embodiment of the present invention, the present invention can be implemented in a digital device, wherein the aforementioned digital device at least comprises a CPU, a memory, an electronic database and a storage unit which are electrically connected to each other. The aforementioned electronic database includes, but not limited to a public health database 21. The aforementioned digital device can include, but not limited to server computer, cluster server, Cloud platform, desktop, laptop computer, notebook computer, network type computer, tablet PC, smart mobile phone and so on.

In another embodiment of the present invention, the input interface can be a keyboard, or a touch screen interface.

In another embodiment of the present invention, the Machine Learning is selected from, but not limited to Supervised Learning, Unsupervised Learning, Semi-supervised learning, Deep Learning, Reinforcement Learning and Ensemble Learning. For example, the aforementioned machine learning method can be selected from 2D law ordering, Logistic Regression, Decision Tree, neural network learning, K-nearest Neighbor and Bayesian decision method, or an arbitrary combination of the aforesaid methods. Added to this, the neural network can be selected from such deep learning network techniques as Convolutional Neural Network (CNN) and Generative Adversarial Network (GAN).

In another embodiment of the present invention, the aforementioned machine learning software or machine learning model in the present invention can be implemented in the form of software program code executed by the processor, wherein a computer language (e.g. Python, C++, Java or Perl) is used, and the conventional or Object-Oriented Technology is used. The software program code can be stored as a series of instructions or commands in a computer readable media for storage and/or transmission, the suitable media include RANI, ROM, magnetic media (hard disk or floppy disk) or optical media (CD or DVD), flash memory and so on. The computer readable media can be any combination of the storage or transmission apparatuses.

In an embodiment of the present invention, the average cancer risk of population, cancer risk with family history of cancer and cancer risk without family history of cancer are selected from the public health database 21 or the disclosed statistical data of cancer risk. Wherein the public health database 21 is selected from, but not limited to the declared statistical data of public health in the SEER (Surveillance, Epidemiology and End Results) database of National Cancer Institute (NCI). Added to this, the public health database can be selected from the declared statistical data of public health in the cancer records and the declared statistical data of public health of cancer patient survival state or metastasis and recurrence state of the Ministry of Health and Welfare.

The aforementioned average cancer risk of population in the present invention is selected from the average cancer risk of all populations, average cancer risk of Asian and Oceanian populations, average cancer risk of American population, average cancer risk of European population and average cancer risk of African population. Wherein the average cancer risk of all populations can be selected from the average cancer risk of the sexuality and the age of all populations. In addition, the average cancer risk of Asian and Oceanian populations, average cancer risk of American population, average cancer risk of European population and average cancer risk of African population can be selected from the average cancer risk of the sexuality and the age of Asian and Oceanian populations, average cancer risk of the sexuality and the age of American population, average cancer risk of the sexuality and the age of European population and average cancer risk of the sexuality and the age of African population. For example, the average cancer risk of the sexuality and the age of Asian and Oceanian populations can be selected from the average cancer risk (%) of males and the age of Asian and Oceanian populations (as shown in Table 1) and average cancer risk (%) of females and the age of Asian and Oceanian populations (as shown in Table 2). Additionally, the cancer risk with/without family history of cancer includes cancer risk with family history of cancer and cancer risk without family history of cancer, it is derived from the disclosed statistical data of cancer risk.

TABLE 1
Statistical data of cancer risk of males and the
age of Asian and Oceanian populations
Male

| | Lifetime cancer | Cancer mortality |
|---|---|---|
| Age | risk (Lifetime) | (Die_cancer) |
| 0 | 34.32 | 20.17 |
| 10 | 34.46 | 20.33 |
| 20 | 34.42 | 20.35 |
| 30 | 34.4 | 20.42 |
| 40 | 34.22 | 20.45 |
| 50 | 33.69 | 20.38 |
| 60 | 31.84 | 19.89 |
| 70 | 26.96 | 18.53 |
| 80 | 18.95 | 15.8 |

TABLE 2
Statistical data of cancer risk of females and the
age of Asian and Oceanian populations
Female

| | Lifetime cancer | Cancer mortality |
|---|---|---|
| Age | risk (Lifetime) | (Die_cancer) |
| 0 | 33.75 | 16.14 |
| 10 | 33.85 | 16.24 |
| 20 | 33.78 | 16.24 |
| 30 | 33.57 | 16.24 |
| 40 | 32.82 | 16.17 |
| 50 | 30.84 | 15.89 |
| 60 | 27.45 | 15.16 |
| 70 | 22.32 | 13.75 |
| 80 | 15.56 | 11.21 |

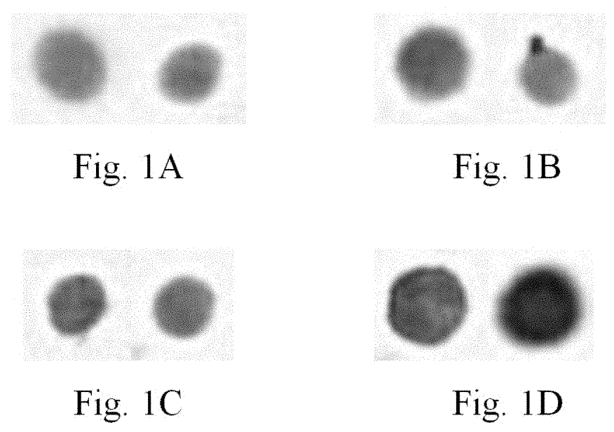

In an embodiment of the present invention, the optical image evaluation and intelligent calibrating system uses immunologic test for GSK-3α antibody to perform immunhistochemical staining of the target cell in the slide, which is scanned by the slide scanner and an image analysis software with machine learning function into an electronic image file. Wherein the aforementioned image analysis software can perform computational analysis with a multitask image analysis module. Furthermore, an analytic parameter of image analysis software is set up, for example, the aforementioned analytic parameter can be a shade threshold, the light-to-deep color grading corresponds to ascending expression level of GSK-3α effectively. In an embodiment of the present invention, the target cell staining results are divided into Grade A (as shown in FIG. 1A), Grade B (as shown in FIG. 1B), Grade C (as shown in FIG. 1C) and Grade D (as shown in FIG. 1D) grading results, and the errors in image analysis software are corrected. The scores of image evaluation results of Grade A, Grade B, Grade C and Grade D of the image analysis software with machine learning function at the boundary condition of pairwise adjacent levels, i.e. the target cells at the boundary edge in the image interpretation results, are definitely compared and classified by manual recheck at initial stage, and the Grade A, Grade B, Grade C and Grade D evaluation results are stored in the image acquisition case database module of optical image evaluation and intelligent calibrating system. For example, Grade A can be defined as a cell/a target cell or a peripheral mononuclear without any GSK-3α protein expression level, Grade D can be defined as the cell/a target cell or the peripheral mononuclear associated with aberrant accumulation of GSK-3α in nucleus. With accumulation of cases of interpretation results and grading results in the case database, the matching, evaluation, interpretation, classification and grading of target cells become more accurate, thus the errors in the course of image capture can be reduced, the efficiency of optical image evaluation and intelligent calibrating system is further increased. In other words, the aforementioned percentages of Grade A, Grade B, Grade C and Grade D of target cell are converted into scores according to proportions, and converted into “system score-1”, which is more accurate.

Further, the present invention is not limited to this, in another embodiment of the present invention, the aforementioned optical image evaluation and intelligent calibrating system can match, evaluate, interpret and classify bone-marrow-derived cells or unhealthy cells through image analysis software to grade GSK-3α protein expression level, a “system score-2” is obtained from the feature of whether GSK-3α protein has excessive expression or accumulates in target cell to the extent of the “unhealthy cell” or “lethal cell” that classified by this invention.

In another embodiment of the present invention, the cancer risk prediction machine learning system will produce a cancer risk prediction table of different cancer risk values of the individual (i.e. cancer-free health estimates of the individual) by obtaining the undermentioned parameters and machine learning and calculation. The parameters are selected from (1) Grade A/Grade B/Grade C/Grade D of GSK-3α protein expression level; (2) with unhealthy cell/without unhealthy cell/with lethal cell/without lethal cell; (3) average cancer risk of population; (4) sexuality; (5) age; (6) cancer risk with family history of cancer/cancer risk without family history of cancer. The aforementioned cancer risk prediction table can be produced and displayed in a display interface or a written report.

In another embodiment of the present invention, an anti-cancer capability level can be divided into Level 1, Level 2, Level 3, Level 4 and Level 5. Each level of the anti-cancer capability level can be divided into a positive grade (+grade/grade+) and a negative grade (−grade grade−). Moreover, a cell interpretation value is derived from the specimen with/without unhealthy cell, wherein cell interpretation value can be divided into a cell interpretation weighted value “Y” and a cell interpretation weighted value “N”, wherein the cell interpretation weighted value “Y” represents the specimen with unhealthy cell as identified by image evaluation and machine learning, the cell interpretation weighted value “N” represents the specimen without unhealthy cell as identified by image evaluation and machine learning.

In other words, a composite index of anti-cancer capability of the present invention can be derived from the aforementioned parameters (1) GSK-3α protein expression level; (2) with/without unhealthy cell through machine learning and calculation. The aforementioned composite index of anti-cancer capability can be divided into 1Y, 2Y, 3BY, 3AY, 3BN, 3AN, 4N and 5N in an embodiment. Wherein the composite index of anti-cancer capability is 1Y>2Y>3BY>3AY>3BN>3AN>4N>5N in ascending order of risk. The A of the aforementioned 3AY represents positive grade, “Y” represents the cell interpretation weighted value Y; the B of the aforementioned 3BN represents negative grade, “N” represents the cell interpretation weighted value N.

In another embodiment of the present invention, the horizontal axis of the aforementioned cancer risk prediction table is the composite index of anti-cancer capability, the vertical axis is the age. In ascending order of cancer risk, the composite index of anti-cancer capability is 1Y, 2Y 3BY, 3AY, 3BN, 3AN, 4N and 5N from left to right. The vertical axis is the age, comprising 20, 30, 40, 50, 60, 70 and 80 years old from top to bottom. Moreover, in another embodiment of the present invention, according to whether there is family history of cancer or not, the cancer risk prediction table can be divided into a cancer risk prediction table without family history of cancer (NCR) (as shown in Table 3, a reference table based on the existing public health data and detection results of the present invention) and a cancer risk prediction table with family history of cancer (CR) (as shown in Table 4). In other words, the lifetime cancer risk decreases as age increases, and the 5N, 4N, 3AN, 3BN, 3AY, 3BY, 2Y and 1Y of composite index of anti-cancer capability represent increasing lifetime cancer risk. Moreover, in another embodiment of the present invention, the cancer risk prediction table can be represented as an abridged table of cancer risk prediction.

TABLE 3
abridged table of cancer risk estimation without
family history of cancer family history of cancer
(NCR), case study of 20 to 80 years old males

| NCR | 1Y | 2Y | 3BY | 3AY | 3BN | 3AN | 4N | 5N |
|---|---|---|---|---|---|---|---|---|
| M20 | 75% | 59% | 45% | 31% | 24% | 18% | 14% | 10% |
| M30 | 75% | 59% | 45% | 31% | 24% | 18% | 14% | 9% |
| M40 | 75% | 59% | 45% | 31% | 24% | 18% | 13% | 9% |
| M50 | 74% | 59% | 44% | 31% | 24% | 18% | 12% | 8% |
| M60 | 69% | 55% | 41% | 28% | 23% | 16% | 11% | 7% |
| M70 | 57% | 45% | 33% | 22% | 17% | 13% | 9% | 6% |
| M80 | 40% | 30% | 20% | 13% | 9% | 7% | 6% | 5% |

TABLE 4
Abridged table of cancer risk estimation without family history
of cancer family history of cancer (NCR), case study with family
history of cancer (CR), case study of 20 to 80 years old males

| CR | 1Y | 2Y | 3BY | 3AY | 3BN | 3AN | 4N | 5N |
|---|---|---|---|---|---|---|---|---|
| M20 | 80% | 69% | 59% | 49% | 44% | 34% | 24% | 15% |
| M30 | 80% | 69% | 59% | 49% | 44% | 34% | 24% | 15% |
| M40 | 80% | 69% | 59% | 49% | 44% | 34% | 24% | 15% |
| M50 | 78% | 68% | 58% | 48% | 44% | 34% | 24% | 14% |
| M60 | 74% | 64% | 54% | 46% | 42% | 31% | 21% | 13% |
| M70 | 66% | 57% | 49% | 41% | 37% | 28% | 20% | 12% |
| M80 | 53% | 45% | 38% | 32% | 29% | 21% | 14% | 8% |

In an embodiment of the present invention, 3BN is selected as data reference of average cancer probability, according to the statistical data of healthy people obtained by the present invention, the grades 1Y, 2Y, 3BY and 3AY of composite index of anti-cancer capability account for 30˜39%, the grades 3AN, 4N and 5N of composite index of anti-cancer capability account for 40˜49%, so the average cancer occurrence probability of overall population falls into 3BN population.

Wherein 3BN (NCR)=National Cancer Institute (NCI) average value of the sexuality and age−difference/2. 3BN (CR)=National Cancer Institute (NCI) average value of the sexuality and age+difference/2. The difference is derived from the statistical data of cancer risk obtained by Big Data calculation and “unhealthy cell” detection information analysis of complete clinically pathological data of the patients with different kinds of cancers.

Further, the aforementioned average cancer probability in the present invention is the statistical data of cancer risk derived from the public health data and clinically pathological data of the patients with different kinds of cancers through calibrating, calculation and fitting, analysis of Big Data. Afterwards, the average cancer risk of families with high cancer risk and the average cancer risk of families with low cancer risk are calculated according to the statistical data of cancer risk. For example, according to the clinical study statistics of targeted protein GSK-3α for high-cancer-risk families and non-high-risk families of healthy people, the “unhealthy cell” status of risk of high-cancer-risk families reflected in clinical study is 2-8 times of that of low-cancer-risk families, after the weighting calculation of cancer risk, it reveals higher cancer occurrence risk of 10% to 30% between high-cancer-risk family and non-high-cancer-risk family under the status of overall average 34%. This information is combined with average cancer occurrence probability and the clinical study result of the present invention, the average cancer probability falls into 3BN population, the average cancer occurrence probability of 3BN population of high-cancer-risk families and non-high-risk families can be set up, and this average cancer probability can be corrected continuously according to the update of declared statistical data of public health in the public health database and the “unhealthy cell”/“lethal cell” detection data accumulation.

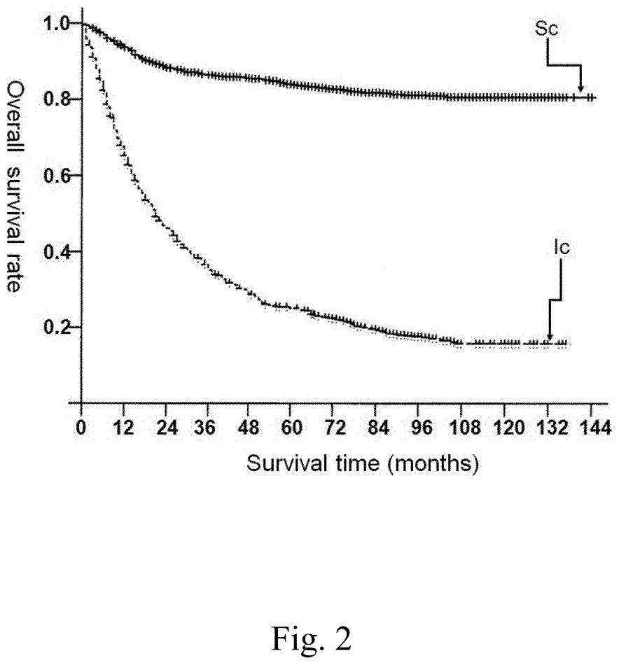

Further, the upper bound of lifetime cancer risk value in Table 3 and Table 4 shall fall on the population of high-cancer-risk family, 20 years old and anti-cancer capability grade 1Y. Referring to the survival curve of GSK-3α in cancer metastasis and recurrence of cancer patients in FIG. 2, the cancerous mechanism of normal persons/persons without cancer in GSK-3α detection is equivalent or similar to the mechanism of cancer metastasis and recurrence of cancer patients in the survival curve. Therefore, the upper bound of cancer risk value is 78%˜85% according to FIG. 2 and statistical detailed information.

The lower bound of lifetime cancer risk value in Table 3 and Table 4 shall fall on the population of non-high-cancer-risk family, 20 years old and grade 5N of composite index of anti-cancer capability (the cancer risk after 20 years old increases but the lifetime risk decreases due to the cancer occurrence people will be deducted from this calculation), the same as the aforesaid estimation mechanism, the lower bound value of cancer risk value is estimated at 10˜16% according to FIG. 2 and statistical detailed information.

The cancer risk estimation table derived from the composite index of anti-cancer capability, age and with/without family history of cancer through machine learning and calculation has 1000˜1200 groups of estimated data according to accuracy requirement. In other words, the present invention uses the average cancer risk of population of the sexuality and the age, cancer risk with family history of cancer and cancer risk without family history of cancer in public health database 21, and uses the bi-directional ranking relation (age related ranking and detection result related ranking) of the cancer risk estimation machine learning system and the set point of average value as the base of correction and estimation for data mining. In addition, in an embodiment of the present invention, the average cancer incidence of the sexuality and the age of Asian and Oceanian populations and cancer risk with family history of cancer or relative cancer risk of cancer risk without family history of cancer are used to perform data separation operation for the average cancer risk of the two populations with family history of cancer and without family history of cancer, so as to calculate the probability and value about cancer risk.

After data separation operation of the average cancer risk of the two populations with family history of cancer and without family history of cancer, the value of the average cancer risk±the difference/2 of average cancer risk between the populations with and without family history of cancer is used as the aforesaid composite index of anti-cancer capability 3BN of the sexuality and the age. The aforementioned average cancer risk can be integrated into the cancer risk estimation machine learning system for data mining, calculation, update and adjustment of declared statistical data of public health as the cancer risk estimation machine learning system of the present invention extracts declared statistical data of public health via the Internet by machine learning.

Wherein the cancer risk with family history of cancer of the individual=average cancer risk of the sexuality and the age+difference/2 of average cancer risk between the populations with and without family history of cancer. In other words, the individual has family history of cancer, the cancer risk with family history of cancer can be derived from average cancer risk of the sexuality and the age+average value of difference between the cancer risk of population with family history of cancer and the cancer risk of population without family history of cancer. Wherein the cancer risk without family history of cancer of the individual=average cancer risk of the sexuality and the age−difference/2 of average cancer risk between populations with and without family history of cancer. In other words, the individual has no family history of cancer, the cancer risk can be derived from the average cancer risk of the sexuality and the age−the average value of difference between the cancer risk of population with family history of cancer and the cancer risk of population without family history of cancer.

However, comparatively, if a person of this field who is familiar with the technology describes an individual's cancer risk only with the existing declared statistical data of public health, the data basis is quite limited. In other words, the existing technology cannot provide the lifetime non-cancer risk estimation value of individuals as accurately as the present invention which uses the composite system of optical image evaluation and intelligent calibrating system and cancer risk estimation machine learning system to obtain the cancer risk estimation table through machine calculation.

To sum up, the aforementioned data are processed by bi-directional ranking relation, Logistic Regression, machine learning and data approaching, the cancer risk analysis ordering and machine learning system of the present invention can import the conditions of different individuals/subjects and detection results into this cancer risk calculation model to generate lifetime cancer risk estimation value or lifetime non-cancer risk estimation value.

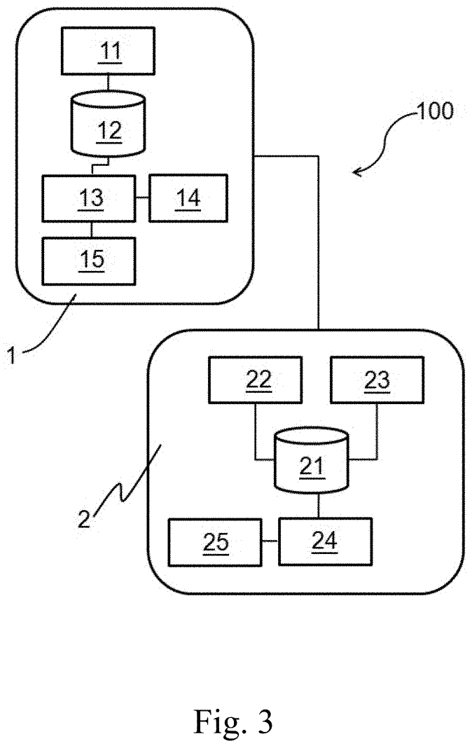

As shown in FIG. 3, the optical image evaluation and intelligent calibrating system 1 of the present invention comprises an image modeling module 11, an image acquisition case database module 12, a multitask image analysis module 13, an image output module 14 and an image setting module 15. The aforementioned optical image evaluation and intelligent calibrating system 1 can be applied to digital devices.

The image modeling module 11 can build an image detection model. In the present invention, the image modeling module 11 usually uses enough images to train the machine learning algorithm applicable to image recognition to build an image detection model. The images used by the image modeling module 11 for building the detection model include the aforesaid target cell image and the grading result images of GSK-3α protein expression levels of Grade A, Grade B, Grade C and Grade D of cell staining results.

The image acquisition case database module 12 can obtain the electronic image files of detection via the image analysis software. The multitask image analysis module 13 can use the image detection model built by image modeling module 11 to analyze the detection images involved in the detection result obtained by image acquisition case database module 12, and the image detection model generates the corresponding analysis result. The multitask image analysis module 13 can provide the detection image obtained by image acquisition case database module 12 as input data for the detection model, so that the detection model analyzes the imported detection model and exports the corresponding analysis result. The analysis result of detection model can indicate whether there is target cell like unhealthy cell and/or GSK-3α protein expression level in the detection image.

The image output module 14 can display the detection image when the detection image contains target cell and/or GSK-3α protein expression level. The image setting module 15 enables the image modeling module 11 to further train the detection model according to the confirmation data set by image setting module 15 and the corresponding detection image, so that the detection model can make more accurate decision.

In other words, after the image modeling module 11 builds the detection model and the image acquisition case database module 12 obtains detection result, the multifunctional image analysis module 13 can use the detection model built by image modeling module 11 to analyze the detection image obtained by image acquisition case database module 12, and generate corresponding analysis result.

In another embodiment of the present invention, the cancer risk prediction and machine-learning system 2 comprises a public health database 21, an input module 22, an information acquisition module 23, a machine learning analysis module 24 and an output module 25. The cancer risk prediction and machine-learning system 2 of the present invention can be installed in single server, cluster server, Cloud platform, desktop, laptop computer, notebook computer, network type computer, tablet PC and smart mobile phone. The input module 22 is communicated with public health database 21. A user can input individual's sexuality and age through the input module 22, which are stored in the public health database 21. For example, the input module 22 generates a webpage interface or an Application Programming Interface (API) for the user to input individual's sexuality and age. The information acquisition module 23 is communicated with the public health database 21. The information acquisition module 23 can extract the average cancer risk of population and cancer risk with family history of cancer/cancer risk without family history of cancer, which are stored in the public health database 21. In an embodiment, the information acquisition module 23 can be a web crawler or a Robotic Process Automation (RPA), so the aforementioned average cancer risk of population or cancer risk with family history of cancer/cancer risk without family history of cancer can be automatically extracted by the web crawler or RPA from Internet or public health database. However, the present invention is not limited to this, in another embodiment, the information acquisition module 23 can be a user interface, and the user can input the aforementioned average cancer risk of population or cancer risk with family history of cancer/cancer risk without family history of cancer through this user interface. Added to this, the information acquisition module 23 can automatically extract the detection image of the individual from the optical image evaluation and intelligent calibrating system 1 through web crawler or RPA, so as to generate the corresponding analysis result.

The machine learning analysis module 24 is communicated with the public health database 21. The machine learning analysis module 24 can generate the corresponding analysis result according to prior or the existing average cancer risk of population and/or cancer risk with family history of cancer/cancer risk without family history of cancer and the detection image from optical image evaluation and intelligent calibrating system 1, so as to perform machine learning and build a cancer risk estimation model.

Moreover, the machine learning analysis module 24 obtains the cancer risk estimation table of the individual by the cancer risk estimation model according to the input sexuality and age of the individual. The aforementioned cancer risk estimation table can provide a lifetime lethal cancer risk estimation value of the individual, lifetime cancer risk estimation value and lifetime non-cancer risk estimation value. On the other hand, the machine learning analysis module 24 can predict the individual's cancer risk estimation value according to at least an expert adjusted parameter. For example, the machine learning analysis module 24 can adjust the cancer risk estimation model according to the accumulation of testing data of the present invention and the data updating of public health database, so as to adjust the optimal trend of cancer risk estimation model.

The output module 25 is communicated with the machine learning analysis module 24. The machine learning analysis module 24 predicts the individual's cancer risk estimation result, the output module 25 exports the cancer risk estimation table to provide references for the user or subject to adjust the cancer risk factor and habits and customs, so as to perform holistic health management, improve individual's body condition and keep away from cancer. In an embodiment, the output module 25 can be a display equipment. Moreover, under the Cloud platform architecture, the output module 25 can be a communication interface, e.g. wired network interface, wireless network interface or mobile communication network interface, sending the cancer risk estimation table to remote user devices, for example, but not limited to display interface, fax and duplicator.

On the other hand, in another embodiment, the average cancer risk of the sexuality and the age of population, the individual with/without a cancer risk with family history of cancer, protein expression level, image evaluation result, system score-1, system score-2, composite level of anti-cancer capability, composite level grade of anti-cancer capability, cell parameter and risk index can include at least an evaluation factor and/or a representative value thereof, and the corresponding weighting.

To sum up, the aforementioned composite system of machine learning image interpretation and cancer risk estimation of the present invention can import different individual/subject conditions and detection results into this cancer risk calculation model according to the aforementioned data and bi-directional ranking relation, Logistic Regression, machine learning and approach, so as to generate lifetime cancer risk estimation value or lifetime non-cancer risk estimation value.

### EMBODIMENTS

### Embodiment 1, Determination of Upper and Lower Bounds of Cancer Risk Value of Cancer Risk Estimation Machine Learning Method

As shown in FIG. 2, the cancer tissue slices of almost 2,000 cancer patients are analyzed by using the biotechnical detection technique that applied in the present invention, wherein the case database of the present invention has tracked cases for over 10 years. According to the evaluation of the data in the case database, the survival rate of the cancer patients with lethal cells or unhealthy cells is lower than 20%, which is about 18%, the individuals have cancer recurrence, metastasis and deterioration within 2˜5 years, as shown in the lower curve Ic. The lower curve Ic can represent cancer-risk-detection (+), the applied diagnostic technique for the present invention has obtained ˜900 cases from the above clinical research data statistics. The survival rate of the cancer patients without lethal cell or unhealthy cell is higher than 80%, which is about 82%, as shown in the upper curve Sc. The upper curve Sc can represent cancer-risk-detection (−), the applied diagnostic technique for present invention has obtained ˜1,100 cases from the above clinical research data statistics. Based on this, the upper bound of cancer risk is larger than 80% according to the lethal cancer risk of high risk group in the present invention, which is about 82%, and the lower bound is set as 5˜8% according to the degree of risk of the cancer-risk-detection (−) group and the lifetime cancer risk decreasing with age. In addition, p<0.001 of upper curve Sc and lower curve Ic, the aforementioned cancer-risk-detection (+) refers to the (individual with) cancer patient's tissue slice containing unhealthy cell with excessive expression of GSK-3α, the cancer-risk-detection (−) refers to the (individual with) cancer patient's tissue slice free of unhealthy cell with excessive expression of GSK-3α. The high risk group refers to the family group with cancer cases within second class relatives. On the other hand, the aforementioned low risk group refers to non-high risk group without the aforementioned family history of cancer.

Further, according to the average lifetime cancer risk of various age-brackets of Asian population of National Cancer Institute, the senior citizens have lower lifetime cancer risk, which is 16˜19%. This lifetime cancer risk is average value, so the actual lifetime cancer risk varies with different factors. The present invention will use the method of approximation or bi-directional ranking relation to gradually approximate the maximum and minimum values of cancer probability and different ages-composite index of anti-cancer capability to the individual's real data through the learning system according to the latest data of public health data published by National Cancer Institute (NCI), Ministry of Health and Welfare (Taiwan) with the cases tested by the present invention, so as to validate the forecast capacity of this model.

### Embodiment 2, Cancer Risk Estimation Table of Case 1

Case 1 is male, 50 years old, with family history of cancer, composite level grade of anti-cancer capability is 4, and the specimen contains unhealthy cells. The lifetime non-cancer risk estimation value of the case obtained by the biotechnical detection technique combined with cancer risk estimation machine learning model of the present invention is 61%.

TABLE 5
Cancer risk estimation table of Case 1
19BXXX2 M50Y_CR

| Anti-cancer capability | Lifetime non-cancer risk | Lifetime cancer risk |
|---|---|---|
| level (grade) | estimation value | estimation value |
| (5Y) | While unhealthy cells exist, there will not be | |
| | anti-cancer capability level 5 | |
| (4Y) | 61% | 39% |
| 3AY | 52% | 48% |
| 3BY | 42% | 58% |
| 2Y | 32% | 68% |
| 1Y | 22% | 78% |

*M50_Ref = 66%

### Embodiment 3, Cancer Risk Estimation Table of Case 2

Case 2 is female, 47 years old, without family history of cancer, composite level grade of anti-cancer capability is 5, and there is no unhealthy cell detected in the specimen. The lifetime non-cancer risk estimation value of the case obtained by the biotechnical detection technique combined with cancer risk estimation machine learning model of the present invention is 92%.

TABLE 6
Cancer risk estimation table of Case 2
19BXXX6 F47N_NCR

| Anti-cancer capability | Lifetime non-cancer risk | Lifetime cancer risk |
|---|---|---|
| level(grade) | estimation value | estimation value |
| 5N | 92% | 8% |
| 4N | 88% | 12% |
| 3AN | 84% | 16% |
| 3BN | 79% | 21% |
| (2N) | 67% | 33% |

| | |
|---|---|
| (1N) | There is no unhealthy cell, there will not be |
| | anti-cancer capability level 1 |

*F47_Ref = 69%

### Embodiment 4, Cancer Risk Estimation Table of Case 3

Case 3 is male, 46 years old, without family history of cancer, composite level grade of anti-cancer capability is 3—(3B), and there is unhealthy cell detected in the specimen. The first lifetime non-cancer risk estimation value of the case obtained by the biotechnical detection technique combined with cancer risk estimation machine learning model of the present invention is 55%. The case is tested for the second time after a period of time, the composite level grade of anti-cancer capability is increased to 4, and there is no unhealthy cell detected in the specimen, the lifetime non-cancer risk estimation value of the case obtained by the biotechnical detection technique combined with cancer risk estimation machine learning model of the present invention is greatly increased to 88%.

TABLE 7
Cancer risk estimation table A of Case 3
M46N_NCR

| Anti-cancer capability | Lifetime non-cancer risk | Lifetime cancer risk |
|---|---|---|
| level (grade) | estimation value | estimation value |
| 5N | 92% | 8% |
| 4N | 88% | 12% |
| 3AN | 82% | 18% |
| 3BN | 76% | 24% |
| (2N) | 62% | 38% |

| | |
|---|---|
| (1N) | There is no unhealthy cell, there will not be |
| | anti-cancer capability level 1 |

*M46_Ref = 66%

TABLE 8
Cancer risk estimation table B of Case 3
M46Y_NCR

| Anti-cancer capability | Lifetime non-cancer risk | Lifetime cancer risk |
|---|---|---|
| level (grade) | estimation value | estimation value |
| (5Y) | While unhealthy cells exist, there will not be | |
| | anti-cancer capability level 5 | |
| (4Y) | 79% | 21% |
| 3AY | 69% | 31% |
| 3BY | 55% | 45% |
| 2Y | 41% | 59% |
| 1Y | 25% | 75% |

*M46_Ref = 66%

Although the invention has been explained in relation to its preferred embodiment, it is to be understood that many other possible modifications and variations can be made without departing from the spirit and scope of the invention as hereinafter claimed.

1. A composite system of image evaluation, machine learning and cancer risk prediction comprises
an optical image evaluation and intelligent calibrating system, which comprises
an image modeling module, building an image detection model with a cell staining image result of GSK-3α a protein expression level of a target cell;
an image acquisition case database module, obtaining a detection image of a detection result;
a multitask image analysis module, using the image detection model built by the image modeling module to analyze the detection image, and the image detection model generates a corresponding analysis result;
a cancer risk prediction and machine-learning system, which comprises
an input module, for a user to input an individual's sexuality and age through a webpage interface or an API, which are stored in a public health database;
an information acquisition module, which can extract an average cancer risk of population and a cancer risk with family history of cancer/a cancer risk without family history of cancer, which are stored in the public health database;
a machine learning analysis module, which is communicated with the public health database, wherein the machine learning analysis module generates the corresponding analysis result according to the average cancer risk of population and/or cancer risk with family history of cancer/cancer risk without family history of cancer and the detection image to perform machine learning and build a cancer risk estimation model, so as to obtain a cancer risk estimation table of the individual;
wherein after data separation operation of the average cancer risk of the two populations with family history of cancer and without family history of cancer, the average cancer risk±difference/2 of average cancer risk between the populations with and without family history of cancer is a composite index of anti-cancer capability 3BN of the sexuality and the age.

2. The system of claim 1, wherein the target cell is selected from an unhealthy cell or a lethal cell.

3. The system of claim 1, wherein the cell staining image result is classified into Grade A, Grade B, Grade C and Grade D.

4. The system of claim 3, wherein the Grade A is defined as a cell without any GSK-3α protein expression level, wherein the Grade D is defined as the overall nucleus of the cell associated with aberrant accumulation of GSK-3α in nucleus.

5. The system of claim 1, wherein the cancer risk of the individual with family history of cancer is equal to the average cancer risk of the sexuality and the age+½ of difference of average cancer risk between the populations with and without family history of cancer.

6. The system of claim 1, wherein the cancer risk of the individual without family history of cancer is equal to the average cancer risk of the sexuality and the age−½ of difference of average cancer risk between the populations with and without family history of cancer.

7. The system of claim 1, wherein the cancer risk estimation table provides a lifetime non-cancer risk estimation value of the individual.
